(19) United States
(12) Patent Application Publication
(10) Pub. No.: US 2012/0215258 A1
(43) Pub. Date: Aug. 23, 2012

(54) Chiropractic distraction plate

(76) Inventors: Harvey J. Segur, Portland, OR (US); Sandra Kay Diehl, (US)

(21) Appl. No.: 12/932,066

(22) Filed: Feb. 17, 2011

(51) Int. Cl. A61F 5/04

(52) U.S. Cl. 606/241

(57) ABSTRACT

A portable devise that provides a plate attached to a ball and socket structure with an adjustable range-of-motion ring supported by a stable rigid platform that is capable of performing gyroscopic motion that can be applied to the spine or pelvis in either the prone, supine, or sitting position when placed on the floor, structure with flat surface, chair, or table to be used in conjunction with the methods, techniques, and procedures of spinal manual manipulation and manipulative surgery by a doctor educated and trained in the professions of either chiropractic, osteopathy, naturopathy, or physical medicine for treatment of spinal and pelvic disorders, conditions, and syndromes.

### BACKGROUND

1. Field

This application relates generally to devices that are used in conjunction with the methods, techniques, and procedures of spinal manual manipulation and manipulative surgery by a doctor educated and trained in the professions of either chiropractic, osteopathy, naturopathy, or physical medicine for treatment of spinal and pelvic disorders, conditions, and syndromes.

2. Prior Art

Heretofore, there have been various devices that have been invented that claim to stretch, traction, decompress, distract, mobilize, and reposition the articular elements of the spine and pelvis that have been designed for self-use, self treatment, or incorporated into a therapeutic table. The concept of the present invention is a portable device that provides a plate attached to a ball and socket structure with an adjustable range-of-motion ring supported by a stable rigid platform that is capable of performing gyroscopic motion that can be applied to the spine or pelvis in either the prone, supine, or sitting position when positioned on the floor, structure with flat surface, chair, or table.

Prior art thought to be most closely associated with the present invention are the inventions disclosed in U.S. Pat. No. 478,166 to Madsen (1892), U.S. Pat. No. 1,011,038 to Davenport (1911), U.S. Pat. No. 1,547,482 to Woodruff (1925), U.S. Pat. No. 1,949,859 to Charles (1932), U.S. Pat. No. 5,152,691 to Moscarello (1992), U.S. Pat. No. 5,328,421 to Stanalajczo (1994), and U.S. Pat. No. 7,137,938 to Gottlieb (2006). The Madsen invention of 1892 was designed to be an exercise apparatus for the feet and not for therapeutic use of the spine and pelvis. The Davenport invention of 1911 demonstrates a gyroscopic motion of a front and rear section of a therapeutic table, but the full gyroscopic capability of each section is restricted by design, a built-in function of the table, and is not portable. The Woodruff invention of 1925 is a universal ball joint, but was invented primarily for railway coupling use and is not adjustable. The Charles invention of 1934 is a universal ball joint, but was invented for the purpose and function of lubricated coupling and is not adjustable. The Moscarello invention of 1992 has limited gyroscopic motion, but was designed as an apparatus that would allow the user to simulate the action of a snowboard during dryland training and not for therapeutic purpose for the spine and pelvis. The Stanalajczo invention of 1994 was designed as a rehabilitation device for the lower extremity with a tipping function without gyroscopic motion. The Gottlieb invention of 2006 has gyroscopic motion, which can be altered and restricted to a pivot about a fulcrum, but was designed to have a person stand and balance on it, but was not designed to be used for the spine and pelvis.

None of the aforementioned inventions provide for a device that is designed to be used in conjunction with the methods, techniques, and procedures of spinal manual manipulation and manipulative surgery by a doctor educated and trained in the professions of either chiropractic, osteopathy, naturopathy, or physical medicine for treatment of spinal and pelvic disorders, conditions, and syndromes.

### SUMMARY

In accordance with the concept of the first embodiment the present invention is a portable devise that provides a plate attached to a ball and socket structure with an adjustable range-of-motion ring supported by a stable rigid platform that is capable of performing gyroscopic motion that can be applied to the spine or pelvis in either the prone, supine, or sitting position when placed on the floor, structure with flat surface, chair, or table.

### DRAWINGS

### Figures

The following is an itemized list of the figures referred to in this patent application.

FIG. 1 shows a perspective view of the distraction plate.

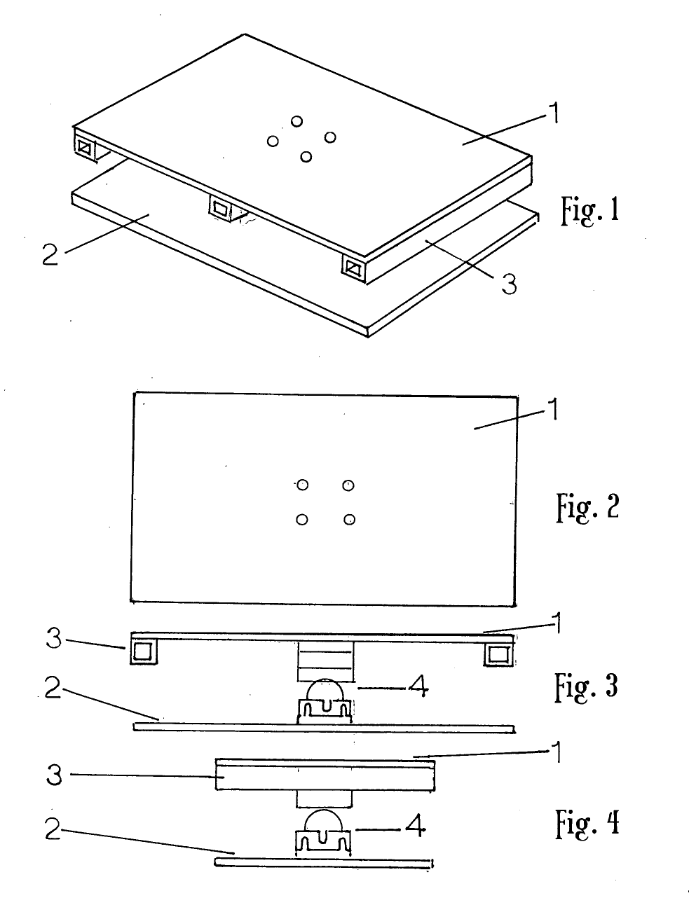

FIG. 2 shows a top sectional view of FIG. 1.

FIG. 3 shows a front sectional view of FIG. 1.

FIG. 4 shows a side sectional view of FIG. 1.

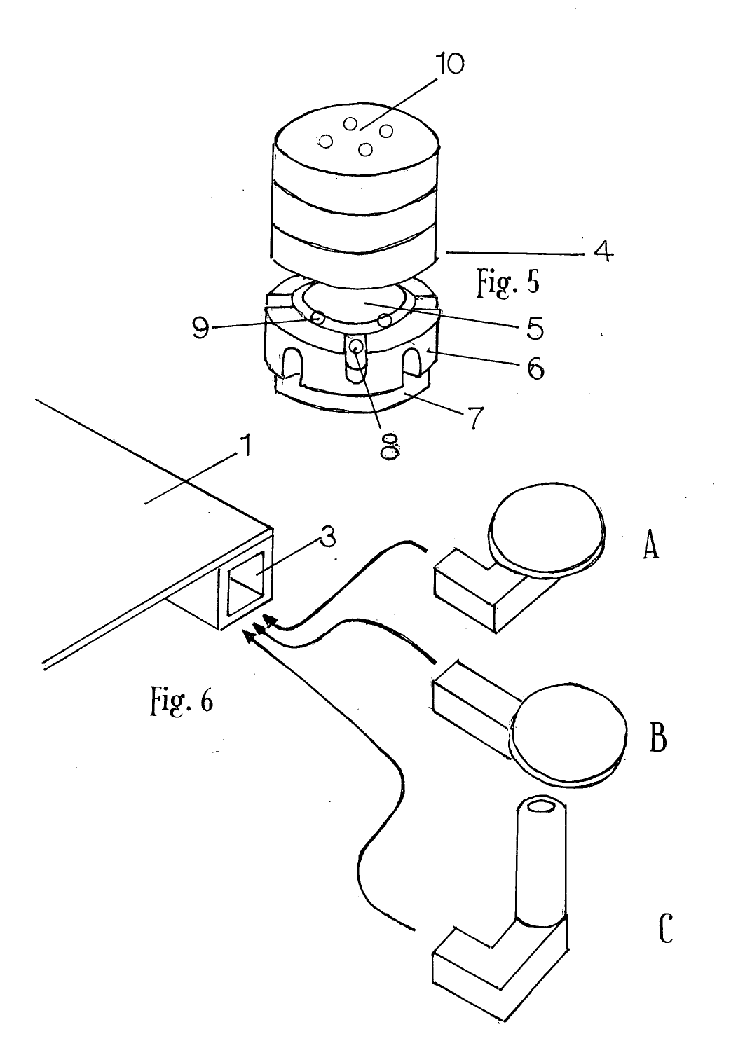

FIG. 5 shows a perspective view of the ball and socket joint mechanism.

FIG. 6 shows a corner sectional view of the plate with attached tube and channel.

### REFERENCE LETTERS

A shows an example of a removable handle with a 90 degree post.

B shows an example of a removable handle with a straight post.

C shows an example of a removable handle of vertical elevation with a 90 degree post.

### REFERENCE NUMBERS

1 Plate.

2 Rigid platform.

3 Tube and channel.

4 Stem receiver.

5 Ball and stem.

6 Adjustable range-of-motion ring.

7 Ball receiver with threaded base.

8 Motion reduction port.

9 Motion reduction screw.

10 Plate attachment mount.

### DETAILED DESCRIPTION OF PREFERRED EMBODIMENTS

As shown in FIG. 1 a portable distraction plate device with a first embodiment consisting of a plate 1 attached to a ball and socket joint structure FIG. 5 with an adjustable range-of-motion ring 6 supported by a stable rigid platform 2.

The plate 1 is illustrated to have a rectangular shape, but other geometric shapes can be used, as long as, adequate support of sections of the spine and/or pelvis can be maintained during treatment. The plate 1 is designed to provide for the mounting of a surface pad for patient comfort during treatment. The plate can be made of any man-made or natural rigid material consisting of either wood, metal, or plastic.

The ball and socket joint structure FIG. 5 consists of a ball and stem 5 residing in a stem receiver 4 and attached to a ball receiver with threaded base 7 with motion reduction screws 9, which includes an adjustable range-of-motion ring 6 with notches that allow access to motion reduction ports 8. The ball and socket joint structure can be made of any man-made or natural rigid material consisting of either wood, metal, or plastic.

The rigid platform 2 is attached to the ball receiver and threaded base 7 as demonstrated in FIG. 3 and FIG. 4 and illustrated to have a rectangular shape, but other geometric shapes can be used, as long as, adequate support of sections of the spine and/or pelvis can be maintained during treatment. Attached to the contact surface of the rigid platform 2 is a non-abrasive skid resistant material to prevent unpredictable movement of the entire device under load of a patient's weight. Even though the concept of the first embodiment stipulates intended use by a doctor educated and trained in the methods, techniques, and procedures of manual manipulation and manipulative surgery this should not be interpreted as a restriction for individual home-use or self-use by prescription for a patient as part of treatment protocol.

Thus the scope of the invention should be determined by the appended claims and their legal equivalents, and not by the examples given.

1. A chiropractic distraction plate consisting of
a. Plate.
b. Tube and channel attached to the sides of the plate.
c. Ball and socket joint structure with an adjustable range-of-motion ring.
d. Rigid platform as base.
e. Portable
f. Handles can be mounted to the tube and channel with retaining device or pin.

2. The concept of the first embodiment of claim 1 is a portable devise that provides a plate attached to a ball and socket structure with an adjustable range-of-motion ring supported by a stable rigid platform that is capable of performing gyroscopic motion that can be applied to the spine or pelvis in either the prone, supine, or sitting position when placed on the floor, structure with flat surface, chair, or table.

3. A device of claim 1 that is to be used in conjunction with the methods, techniques, and procedures of spinal manual manipulation and manipulative surgery by a doctor educated and trained in the professions of either chiropractic, osteopathy, naturopathy, or physical medicine for treatment of spinal and pelvic disorders, conditions, and syndromes.

4. A device of claim 1 that can produce hemispheric gyroscopic motion above the base platform in which a geometric cone of biomechanical effect is produced in 360 degrees of rotation.

5. A function of the device of claim 1 with consideration of claim 3 allows for the potential of rehabilitation exercises to be performed on said device under the direction and supervision of the doctor.

6. A function of the device of claim 1 with consideration of claim 3 allows for the application of the methods and techniques of spinal distraction which is the procedure of separating contiguous articular surfaces by combining segmental axial traction with the motions of flexion, extension, lateral flexion, or circumduction with or without body retraining belt or strap attached to plate.

7. A function of the device of claim 1 in consideration of claim 6 allows for the application of of the methods and techniques of spinal distraction in either a caudad or cephalad direction.
